(19) United States
(12) Patent Application Publication
(10) Pub. No.: US 2024/0355227 A1
(43) Pub. Date: Oct. 24, 2024

(54) Artificial Calculus

(71) Applicant: Regents of the University of Minnesota, Minneapolis, MN (US)

(72) Inventors: Alex Siu Lun Fok, Plymouth, MN (US); Anqi Zhang, Chandler, AZ (US); Ruoqiong Chen, Woodbury, MN (US); Tamer Abdelrehim, Saint Paul, MN (US); Bruno Pierre Lima, Minneapolis, MN (US); Stephanie Porter, Davis, CA (US); Hooi Pin Chew, Bloomington, MN (US); Dina Elsherbini, St Paul, MN (US); Saba Tohidkhah, St Paul, MN (US)

(73) Assignee: Regents of the University of Minnesota, Minneapolis, MN (US)

(21) Appl. No.: 18/643,653

(22) Filed: Apr. 23, 2024

Related U.S. Application Data
(60) Provisional application No. 63/497,903, filed on Apr. 24, 2023.

(51) Int. Cl. G09B 23/28

(52) U.S. Cl. CPC G09B 23/283

(57) ABSTRACT

Example artificial dental calculus models are described. In one example, an in vitro artificial dental calculus includes a dental substrate and a mineralized biofilm comprising an extracellular support material and at least one bacterial species. In one example, a method for creating an in vitro dental calculus model includes applying saliva to a dental substrate in a production environment, applying a mixture of an extracellular support material and at least one bacterial species to the dental substrate, applying a solution comprising calcium and phosphate to the mixture on the dental substrate, and removing the dental substrate from the production environment after a period of time during which a mineralized biofilm comprising the extracellular support material and the at least one bacterial species forms on the dental substrate.

### CROSS REFERENCE TO RELATED APPLICATIONS

This application claims the benefit of U.S. Provisional Patent Application No. 63/497,903, filed on Apr. 24, 2023 and titled “Artificial Calculus”. The entire content of this application is incorporated by reference.

### TECHNICAL FIELD

This disclosure generally relates to artificial calculus and methods for creating artificial calculus.

### BACKGROUND

Dental calculus is the result of mineralization of bacterial plaque on a tooth. Calculus can be a major oral health problem for several reasons. For example, calculus increases the accumulation of plaque and bacterial toxins while also impeding elimination of the plaque and bacterial toxins due to surface roughness of the plaque and calculus. The process continues and can further prevent effective oral hygiene. Calculus treatments, such as methods to remove calculus from a tooth, are typically assessed via clinical studies.

### SUMMARY

Example artificial dental calculus models and methods for generating such dental calculus models are described herein. An artificial dental calculus model may include the generation of dental calculus in an in vitro environment such that the dental calculus can be similar to naturally occurring dental calculus. However, the artificial dental calculus described herein can be formed in much less time than natural dental calculus. Therefore, the artificial dental calculus described herein may provide a medium for testing the prevention or treatment of dental calculus or other processes related to oral health.

The artificial dental calculus may include a mineralized biofilm that includes an extracellular support material, such as collagen, mucin, or other organic compounds, and at least one bacterial species. The mineralized biofilm may be formed on a dental substrate, which may be a natural tooth or a hydroxyapatite layer, for example. In some examples, the process of creating the mineralized biofilm may include several cycles of applying a mixture of the extracellular support material and the at least one bacterial species. The entire process may be completed within several hours or days.

In one example, a method for creating an in vitro dental calculus model, the method comprising applying saliva to a dental substrate in a production environment; applying a mixture of an extracellular support material and at least one bacterial species to the dental substrate; applying a solution comprising calcium and phosphate to the mixture on the dental substrate; and removing the dental substrate from the production environment after a period of time during which a mineralized biofilm comprising the extracellular support material and the at least one bacteria species forms on the dental substrate.

In another example, an in vitro dental calculus model comprises a dental substrate and a mineralized biofilm comprising an extracellular support material and at least one bacterial species.

In another example, a method for creating an in vitro dental calculus model includes applying saliva to a dental substrate for a duration from 1 minute to 6 hours in a production environment, wherein the dental substrate comprises a canine tooth removed of organically deposited plaque, performing at least three cycles that each have a cycle period of at least 48 hours, wherein each cycle of the at least three cycles comprises: applying a mixture of collagen and two bacterial species to the dental substrate, wherein the two bacterial species comprises Neisseria canis and Frederiksenia canicola; applying a solution comprising calcium and phosphate to the mixture on the dental substrate; and removing the dental substrate from the production environment after a period of time during which a mineralized biofilm comprising the collagen and the two bacterial species forms on the dental substrate.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1A includes top views of a in vivo 3D Optical Coherence Tomography (OCT) scan of example canine teeth with naturally occurring calculus.

FIG. 1B includes OCT cross-sectional views of the example teeth of FIG. 1A.

FIG. 2A is an OCT cross-sectional view of an example sectioned canine tooth with overlying artificial dental calculus.

FIG. 2B is an OCT 3D render of artificial calculus digitally segmented from the surface of a tooth.

FIGS. 3A and 3B include perspective views of 3D renders for two different artificial dental calculus models after each cycle of mineralized biofilm growth.

FIG. 4A is an OCT 2D segmented calculus map of an artificial dental calculus model.

FIG. 4B is an OCT 3D segmented calculus map of a top surface of the artificial dental calculus model of FIG. 4A.

FIG. 5A is a graph of natural calculus thickness observed on different teeth.

FIG. 5B is a graph of artificial calculus thickness for different artificial dental calculus models.

FIGS. 6A and 6B are stereomicroscope images of example teeth with natural calculus.

FIGS. 7A, 7B, and 7C are stereomicroscope images of example natural (FIGS. 7A, 7B) and artificial calculus (FIG. 7C) samples.

FIG. 8 includes scanning electron microscope (SEM) images of example natural and artificial calculus samples.

FIG. 9A is a diagram of a crack that can occur within calculus.

FIGS. 9B and 9C are images of example cracks in respective different artificial calculus samples.

FIG. 10 is a flow diagram of an example technique for creating an artificial dental calculus model according to this disclosure.

### DETAILED DESCRIPTION

The disclosure describes artificial dental calculus models and methods for generating such dental calculus models. Dental calculus is a primary contributor to periodontal diseases in both humans and animals. Although calculus treatments can be developed, current assessment of any treatments for preventing or removing dental calculus relies heavily on clinical studies.

Clinical studies can be time-consuming and expensive. For example, natural plaque (biofilms) and calculus (mineralized biofilms) take time to grow. Natural calculus growth is generally limited by the infrequent intake of food and limited supply of minerals in the saliva. In addition, with the large variations in size and chemical compositions and distribution of natural calculus, many subjects (e.g., calculus samples) are required to obtain the data necessary to determine whether or not a treatment for the removal or prevention of calculus is effective using a clinical study.

As described herein, an artificial dental calculus model can be generated in vitro to be substantially similar to natural calculus. The techniques enable a relatively fast growing and reliable artificial dental calculus model that can be generated within a laboratory instead of live subjects. Instead of waiting for the plaque or biofilm (bacteria with extracellular matrix) to grow as done in natural subjects for a clinical study, one example technique includes placing a mixture of collagen and bacteria on tooth samples to preform the plaque. This artificial plaque can then be continuously mineralized using a stabilized calcium phosphate solution to speed up the formation of calculus. In some examples, the calcium and phosphate solution may include a stabilizing agent. The stabilizing agent may include agents such as polyacrylic acid and/or other chemicals. In some examples, a stabilizing agent may include molecules that can inhibit crystallization. In some examples, the stabilizing agent may include molecules that can sequester crystal-forming ions to inhibit crystallization. Other stabilizing agents may include polyaspartic acid, polyallylamine hydrochloride, a polypeptide, as well as natural proteins such as statherin and fetuin and/or other chemicals or compounds.

In some examples, the artificial dental calculus may include a mineralized biofilm that includes an extracellular support material, such as the collagen, mucin or other organic compounds, and at least one bacterial species. The selected bacterial species may be “early colonizers” compared to other bacteria in order to support fast growth of the calculus. The mineralized biofilm may be formed on a dental substrate, which may be a natural tooth or a hydroxyapatite disc, for example. In some examples, the process of creating the mineralized biofilm may include several cycles of applying the mixture of the extracellular support material and the at least one bacterial species. The entire process may be completed within several hours or days.

The methods described herein can produce artificial but clinically representative calculus of the required thickness consistently in a timeframe of hours or days rather than several weeks required by natural methods observed clinically. This artificial dental calculus can then be generated in a much shorter time and without the need for live subjects, which can significantly reduce the time and cost for assessing dental treatments designed for removing and/or preventing calculus. The artificial dental calculus described herein may thus provide a medium for testing treatments or other processes related to oral health. For example, for prevention, a preventative treatment can be applied to a substrate before the substrate is exposed to the artificial calculus creation techniques described herein or the preventative treatment can be applied to the substrate together with (e.g., at the same time as) the artificial calculus creation techniques described herein. The amount of calculus, or lack thereof, may indicate the effectiveness of the preventative treatment.

The examples described herein are directed to canine, or dog, teeth and artificial dental calculus models compared to natural calculus on dog teeth. However, the artificial dental calculus models herein may be similar to other animal teeth, such as other household pets or even humans. Therefore, the artificial dental calculus models and methods to create such models may be similar to those for other teeth and used for assessing calculus treatments for a variety of species.

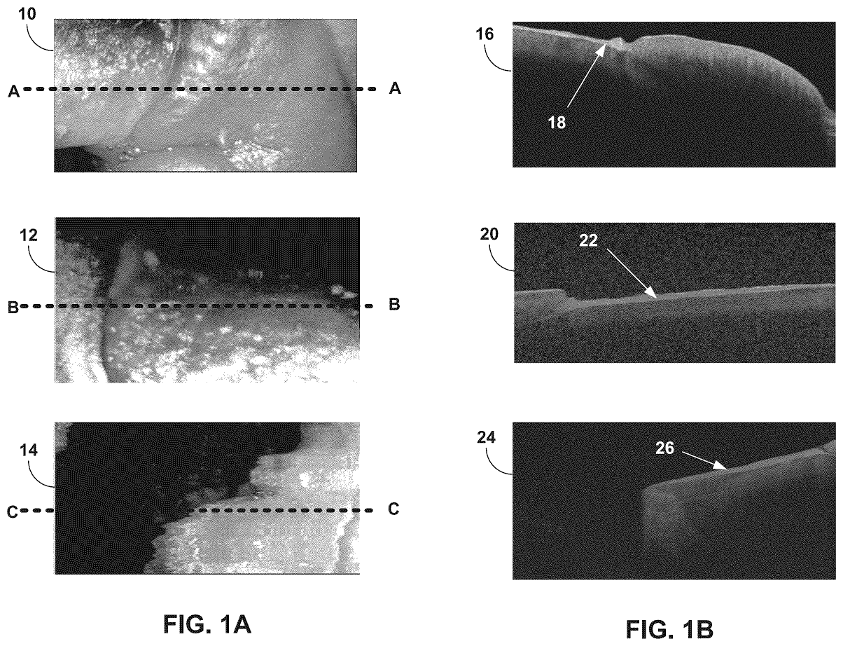

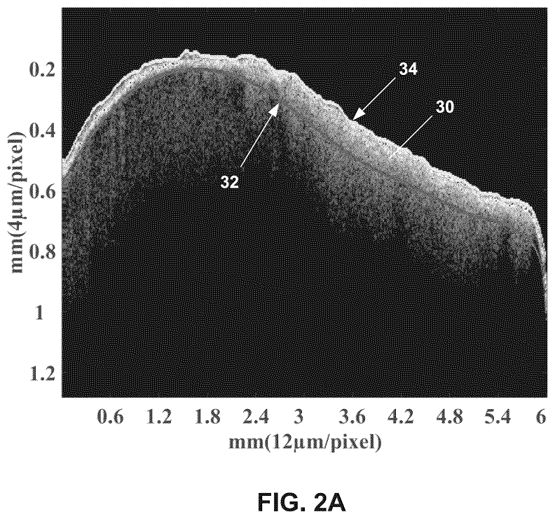

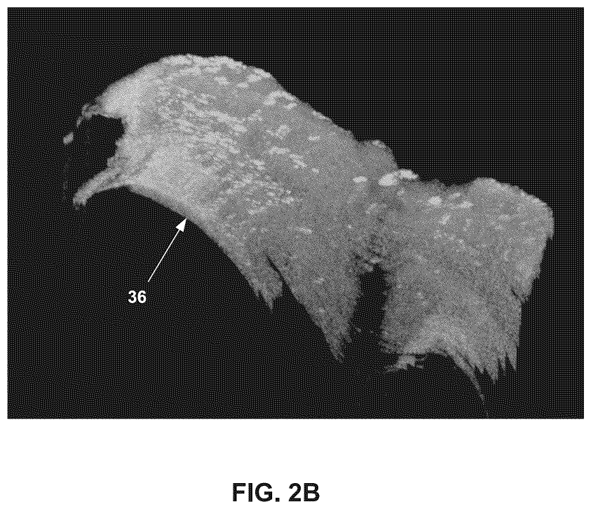

In order to determine if an artificial dental calculus model can be representative of natural calculus, the natural calculus can be characterized as shown FIGS. 1A-2B. The natural calculus characterization can then be used to indicate whether the artificial dental calculus model can be used in place of natural calculus for assessing treatment.

FIG. 1A includes top views of in vivo 3D OCT scans of example teeth with calculus. FIG. 1B includes cross-sectional views of the example teeth of FIG. 1A. Several sample teeth with naturally occurring calculus can be assessed. In the example of FIGS. 1A and 1B, an example dog was allowed to grow calculus for a period of 28 days. After this period of time, several teeth were scanned in order to characterize the calculus growth on the sample teeth of the example dog.

In the example of FIG. 1A, 3D scans 10, 12, and 14 of respective teeth indicate the surface of the tooth. 3D scans 10 and 12 are of respective premolar teeth, and 3D scan 14 is of the left Mn 3^{rd} premolar. Planes A, B, and C indicate the cross-sectional direction that was used to produce each corresponding cross-sectional scans 16, 20, and 24, respectively. Arrows 18, 22, and 26 point to the layer of naturally occurring calculus that is present on the surface of the respective teeth in scans 16, 20, and 25.

FIG. 2A is a cross-sectional view of an example sectioned canine tooth with artificial dental calculus. As shown in the example of FIG. 2A, the cross-sectional view shows the artificial calculus 30 as the lighter shaded areas. The enamel-calculus interface is indicated by the lower line 32, and the exterior surface of the calculus is shown by the upper line 34. The distance between lower line 32 and upper line 34 represents the thickness of calculus 30. This digital segmentation technique of the 2D image can be performed for any sample tooth in order to determine the thickness of artificial or natural calculus on a tooth, such as the values determined in Table 1 below. FIG. 2B is a 3D segmented calculus map of a top surface of a tooth. Calculus 36 is shown to indicate the 3D surface topology.

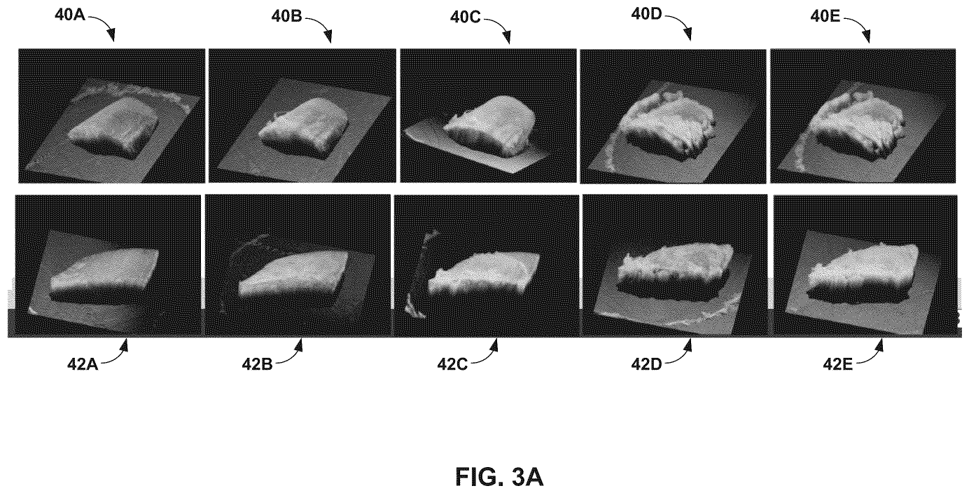

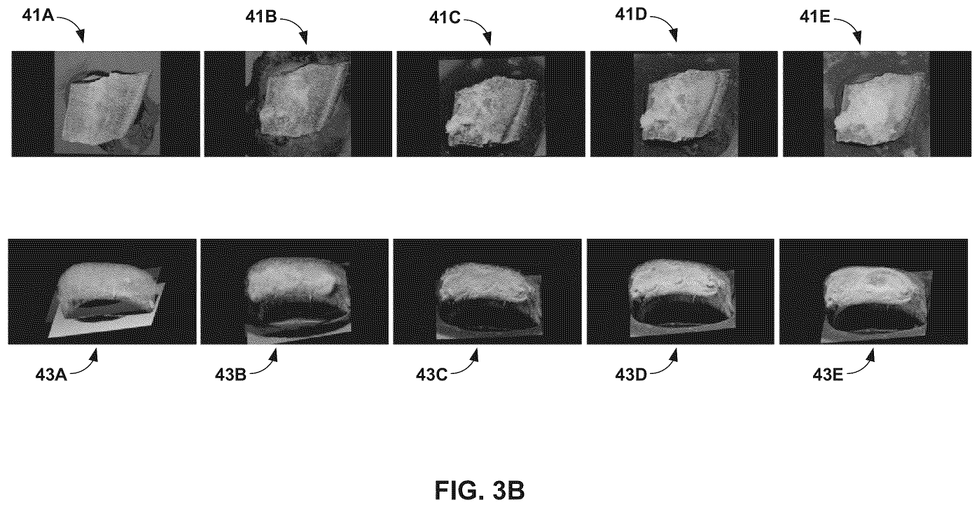

FIGS. 3A and 3B include perspective views of 3D renders for two different artificial dental calculus models (S1 and S5) and (S11 and S15) from two respective batches of mineralized biofilm growth protocol as described in Table 2. As shown in the example of FIG. 3A, a dental substrate was used for each of the samples S1 and S5 and then subject to a mineralizing solution and a mixture of an extracellular support material and bacteria in multiple cycles (40A-40E for sample S1 and 42A-42E for sample S5) in order to grow the artificial dental calculus illustrated in the 4th cycle images for each sample. Each iterative cycle, from the 1^{st} through 4^{th} cycle, applied canine saliva on the dental substrate for a period of time and added additional extracellular support material (e.g., collagen) and bacteria to the sample, the mineralized biofilm appears to increase in thickness and density. Both of samples S1 and S5 were subject to the Batch17 protocol as described below in Table 2.

As shown in the example of FIG. 3B, a dental substrate was used for each of the samples S11 and S12 and then subject to a mineralizing solution and a mixture of an extracellular support material and bacteria in multiple cycles (41A-41E for sample S11 and 43A-43E for sample S12) in order to grow the artificial dental calculus illustrated in the 3rd cycle images for each sample. Each iterative cycle, from the 1^{st} through 3^{rd} cycle, applied artificial saliva on the dental substrate for a period of time and added additional extracellular support material (e.g., collagen, mucin, etc.) and bacteria to the sample, the mineralized biofilm appears to increase in thickness and density. Both of samples S11 and S12 were subject to the Batch29 protocol as described below in Table 2.

Various methods may be used to generate the artificial dental calculus described herein. These artificial dental calculus may be described as in vitro because the calculus is generated within a laboratory setting as opposed to within a living animal. These methods may vary by certain aspects, such as dental substrate, specific bacterial species, the extracellular support matrix used, the types of saliva or cycles and times for each cycle. In one example, method for creating an in vitro dental calculus model includes applying natural or artificial saliva to a dental substrate in a production environment, applying a mixture of an extracellular support material and at least one bacterial species to the dental substrate, applying a solution comprising calcium, phosphate, and, in some examples, a stabilizing agent (e.g., polyacrylic acid and/or another chemical) to the mixture on the dental substrate, and removing the dental substrate from the production environment after a period of time during which a mineralized biofilm comprising the extracellular support material and the at least one bacteria species forms on the dental substrate.

The production environment may refer to a laboratory setting or controlled location where the different components of the technique may be applied to the dental substrate. The production environment may include a bioreactor or similar enclosures that may be subject to certain environmental controls, such as controlled temperature and/or humidity. The production environment would be different than an in vivo environment within a living organism.

In some examples, the dental substrate can be a canine tooth removed of organically deposited plaque. In other words, a natural canine (or dog) tooth may be removed from a dog and then cleaned or stripped of organically deposited (e.g., natural) plaque and calculus. This would form a clean enamel surface at which the artificial calculus can be created. In other examples, artificial dental substrates may be used, such as one or more hydroxyapatite layers.

The saliva may be actual canine saliva or saliva of the intended animal for which the artificial calculus is intended to represent natural calculus. In some examples, artificial saliva may be used, which includes inorganic and or organic components similar to that of natural saliva. In some examples the artificial saliva may be a buffer of one or more chemicals. Artificial saliva may include one or more inorganic compounds such as potassium chloride, sodium chloride, magnesium chloride, calcium chloride, dipotassium phosphate, or monopotassium phosphate. The artificial saliva may also include one or more organic compounds such as proteins and mucins. In some examples, applying the saliva may include applying the saliva to the dental substrate for a duration from a 1 minute to 6 hours. In some examples, the saliva may be applied to the dental substrate for a duration from about 30 minutes to 90 minutes.

In some examples, the process of creating artificial calculus may include one or more cycles of all or some of the steps of the process. In one example, a cycle may include applying the saliva to the dental substrate for a duration of a few minutes up to 6 hours followed by adding the mixture of an extracellular support material and at least one bacterial species and applying the solution of calcium, phosphate, and, in some examples, a stabilizing agent. In some examples, the calculus may be created by performing this cycle of applying the mixture and the solution at least three times. Each cycle may take a period of time that may be at least one hour, at least 12 hours, at least 24 hours, at least 48 hours, or at least 72 hours. Each cycle may have the same duration or different durations.

The extracellular support material may include any material that can provide support for the bacteria to generate an extracellular matrix that becomes part of the mineralized biofilm. Extracellular support materials may include one or more types of proteins or glycoproteins or polysaccharides. In one example, the extracellular support material may include collagen. In another example, the extracellular support material may include mucin, and in some examples, collagen as well. In any of these examples, the extracellular support material may include glycoproteins or polysaccharides. The extracellular support material may include any one or combination of the examples described herein.

The species of bacteria used to create the mineralized biofilm may be important to achieving relatively short times for generating the artificial calculus compared to natural calculus. In some examples, the at least one bacterial species of the oral microbiome (in this case, canine oral microbiome) includes at least two bacteria species. However, that oral microbiome may include three or more different species of bacteria in other examples. However, in some examples, no more than two different bacterial species may be used for creating the calculus. In some examples, no more than four different bacterial species may be used for creating the calculus. In one example, the bacterial species used to create the artificial calculus may include Neisseria canis and Frederiksenia canicola. These specific species of bacteria may work quickly to colonize the dental substrate. In this manner, some example bacterial species may include bacteria that would be classified as an early colonizer (but not limited to) selected to create an extracellular matrix of the mineralized biofilm. Other examples of bacterial species may include Fusobacterium nucleatum and Porphyromonas gulae. Another example may include whole biofilm collected directly from teeth in the canine mouth. Any of these bacterial species, alone or in any combinations thereof, may be used in the methods described herein.

An in vitro dental calculus model may thus include a dental substrate and a mineralized biofilm comprising an extracellular support material and at least one bacterial species. The dental substrate may include a canine tooth removed of organically deposited plaque or an artificial hydroxyapatite layer. The extracellular support material may include collagen in some examples. The at least one bacterial species may include at least two bacterial species, followed by one of more different species. In one example, the bacterial species includes Neisseria canis and Frederiksenia canicola. In one example, the bacterial species includes Neisseria canis and Frederiksenia canicola in the initial period of incubation followed by another period of incubation of Fusobacterium nucleatum and Porphyromonas gulae. In this manner, different bacterial species may be used in different iterations of the application of bacterial species. The bacteria may include an early colonizer selected to better create an extracellular matrix of the mineralized biofilm and followed by bacterial species to agglomerate the mineralized biofilm. As discussed in more detail below, the mineralized biofilm of the artificial dental calculus may have an average thickness greater than 150 micrometers.

Once the artificial dental calculus has been created, it can be used to assess the efficacy of one or more calculus treatments. For example, the method of assessment may include applying a dental treatment to the mineralized biofilm on the dental substrate, and then analyzing an effectiveness of the dental treatment in removing at least a portion of the mineralized biofilm from the dental substrate. Example dental treatments may include chemical selected to dissolve or otherwise remove the artificial calculus from the dental substrate and/or mechanical methods for removing the artificial calculus. Other example of a method of assessment may include applying a dental treatment simultaneously with biofilm mineralization, and then analyzing how the progression of the biofilm mineralization is affected.

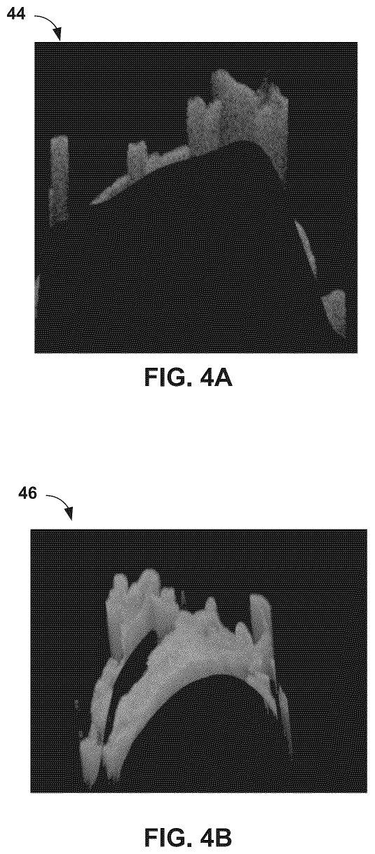

FIG. 4A is a 2D segmented calculus map of a top surface of an artificial dental calculus model. Image 44 of FIG. 4A indicates a 2D segmented cross-section of the 4^{th} cycle of sample S1 of FIG. 3, where calculus is the lighter shades of image 44. FIG. 4B is a 3D segmented calculus map of a top surface of the artificial dental calculus model of FIG. 4A.

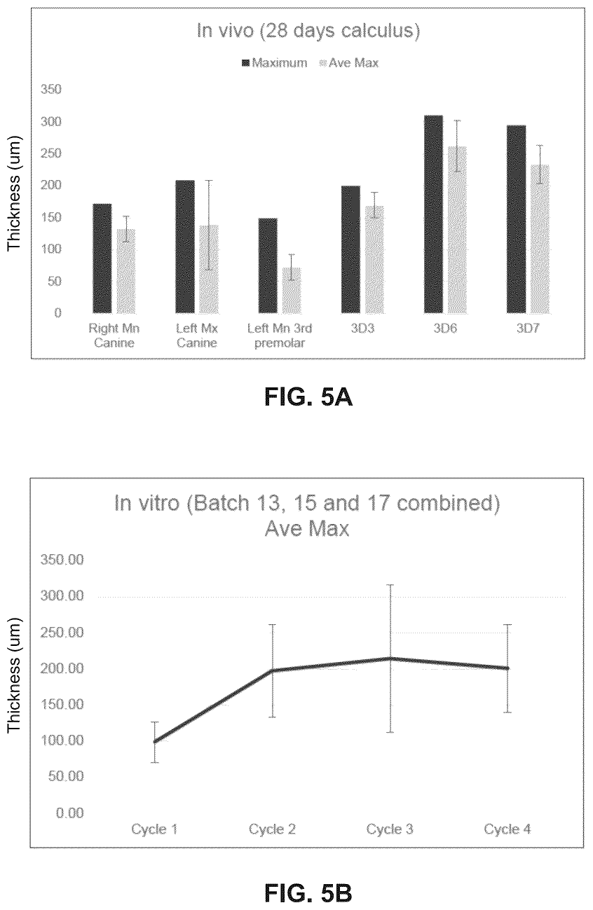

FIG. 5A is a graph of naturally occurring calculus thickness for different teeth. The thickness of the natural calculus in the example teeth was measured via the cross-sectional scans and presented below in Table 1.

TABLE 1

| | Peak Maximum | Average Maximum | Standard |
|---|---|---|---|
| Sample | Thickness (um) | Thickness (um) | Deviation (um) |
| Right Mn Canine | 171 | 132 | 20 |
| Left Mx Canine | 208 | 138 | 70 |
| Left Mn 3^{rd} | 148 | 71.6 | 20 |
| premolar | | | |
| 3D3 | 199 | 169 | 20 |
| 3D6 | 310 | 262 | 40 |
| 3D7 | 294 | 233 | 30 |

As shown in Table 1 above, six different teeth were measured and the peak maximum (located at one of the cross sections) and average maximum (over all cross sections) thickness (in micrometers) are presented in the respective column for each tooth. The standard deviation was calculated for the multiple measurements of each tooth from which the average maximum thickness was determined. The values of Table 1 are illustrated in the graph of FIG. 5A.

Several different techniques for generating the artificial dental calculus were assessed. Some different techniques for generating artificial dental calculus are described in Table 2 below. Each method is labeled as a different “batch,” in which some different aspects of the method are modified. Resulting thicknesses of each artificial calculus are also measured and provided for each method.

TABLE 2

| | Batch11 | Batch12 | Batch13 | Batch15 | Batch16 | Batch17 | Batch 29 |
|---|---|---|---|---|---|---|---|
| Biofilm | 2 species | 2 species | 2 species | 2 species | 2 species | 2 species | 2 species |
| Additive | 1% | 10% | Type I | Type I | Type I | Type I | Type I |
| | mucin | mucin | collagen | collagen | collagen | collagen | collagen |
| | | | solution | solution | solution | solution | solution |
| Substrate | canine | canine | canine | canine | canine | canine | canine |
| | enamel | enamel | enamel | enamel | enamel | enamel | enamel |
| Saliva | no | no | 4 hrs at | 4 hrs at | 4 hrs at | 4 hrs at | 40 minutes |
| coating | | | beginning | beginning | each cycle | beginning | at each cycle |
| Type of | NA | NA | Canine | Canine | Canine | Canine | Artificial |
| saliva | | | saliva | saliva | saliva | saliva | saliva |
| | | | | | | | (added |
| | | | | | | | mucin) |
| # of cycles | 3 | 3 | 4 | 4 | 3 | 4 | 3 |
| # of days per | 2 | 2 | 3 | 3 | 3 | 3 | 3 |
| cycle | | | | | | | |
| Peak Max | 211 | 174 | 371 | 257 | 228 | 548 | 260 |
| Thickness | | | | | | | |
| (um) | | | | | | | |
| Mean max | 152 | 130 | 240 | 206 | 207 | 408 | 220 |
| thickness | | | | | | | |
| (um) | | | | | | | |

As shown in Table 2, Batch13, Batch15, and Batch17 are essentially the same method. The 2 species of bacteria used for all batches in Table 2 were Neisseria canis and Frederiksenia canicola. However, other bacterial species may be used in other examples. In Batch11 and Batch12, no saliva was used over the dental substrate. Saliva was used in each other batch as shown. For Batch13, Batch15, and Batch17, the saliva was only introduced at the beginning before the first cycle of collagen and bacteria. However, for Batch16, saliva was added at the beginning of each cycle. As seen from the peak maximum and average maximum thicknesses of calculus in Table 2, Batch13, Batch15, and Batch17 produced the thickest artificial calculus of the different methods.

FIG. 5B is a graph of artificial calculus thickness for some different generated samples. In particular, the average maximum thickness (in micrometers) for the samples from Batch13, Batch15, and Batch17 is shown over the course of different cycles. The average maximum thickness of the calculus increases from the first to second cycle, but generally leveled off between cycles 2, 3, and 4. In some examples, as few as two cycles may be sufficient to achieve artificial calculus thickness similar to natural calculus. Even though the average maximum thickness may not have increased after cycle 2, the overall density of the artificial calculus may have been improved after cycles 3 and 4. For example, additional cycles may increase the density of the artificial calculus to be closer to naturally occurring calculus.

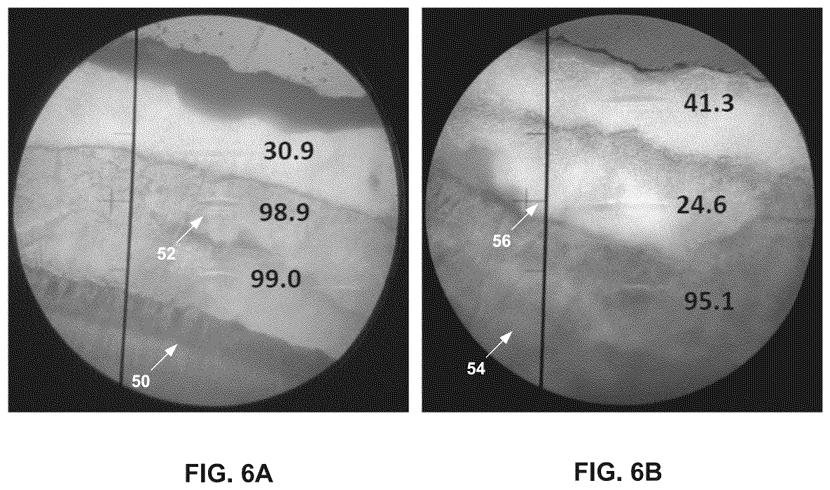

FIGS. 6A and 6B are stereomicroscope images of example teeth with natural calculus. As shown in FIG. 6A, enamel 50 is shown at the bottom, with natural calculus 52 deposited on top of the surface of enamel 50. The hardness values of the natural calculus (Knoop hardness value, 5 g load, 10 s dwelling time) of 99.0, 98.9, and 30.9 (closest to enamel to furthest to enamel respectively) indicate that the harder portions of calculus 52 are deposited closest to the enamel surface. As shown in FIG. 6B, enamel 54 is shown at the bottom, with natural calculus 56 deposited on top of the surface of enamel 54. The hardness values of the natural calculus (Knoop, 5 g, 10 s) of 95.1, 24.6, and 41.3 (closest to enamel to furthest to enamel respectively) again indicate that the hardness values are generally greater for the bottom layers of the calculus 56, but that less hard, or softer, areas of the natural calculus can be interspersed in some areas.

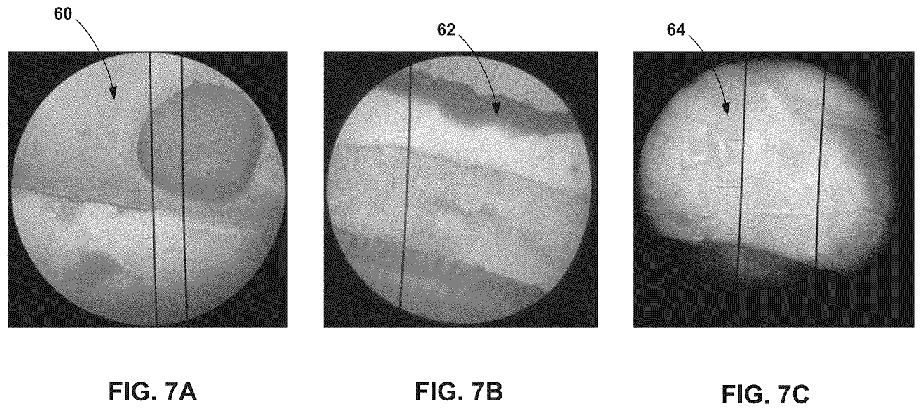

FIGS. 7A, 7B, and 7C are images of example natural (FIGS. 7A and 7B) and artificial calculus (FIG. 7C) samples. Natural calculus 60 of FIG. 7A is relatively old (more than a year) and thick, which may have an average hardness (Knoop hardness value, 5 g load, 10 s dwelling time) of approximately 88.0 with a standard deviation of 23.6 in some examples. Natural calculus 62 of FIG. 7B is younger (between 1-3 months), thinner, and has a lower hardness level, which may have an average hardness (Knoop, 5 g, 10 s) of approximately 54.8 with a standard deviation of 28.4 in some examples. An example artificial calculus 64 of FIG. 7C is relatively new by the nature of the methods described herein, which may have an average hardness (Knoop, 5 g, 10 s) of approximately 21.6 with a standard deviation of 5.0 in one example. These hardness examples indicate that artificial calculus has lower hardness values than natural calculus and the hardness values are closer to that of young natural calculus or in some cases the surface of old natural calculus. Therefore, the artificial calculus may function as a representation similar to young natural calculus for the purposes of evaluating calculus treatment efficacies.

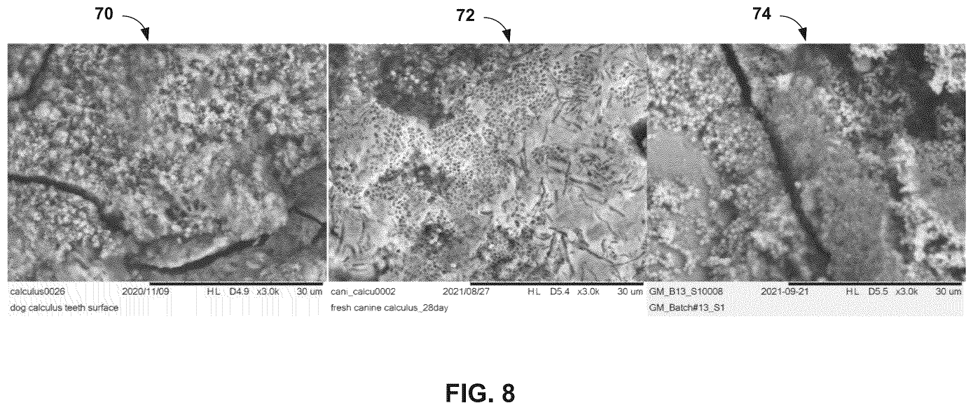

FIG. 8 includes scanning electron microscope (SEM) images of example natural and artificial calculus samples. Image 70 is of natural canine calculus, and image 72 is of natural canine calculus after 28 days of growth. The surface patterns of the calculus in images 70 and 72 for natural calculus is similar to the surface patterns of image 74 of artificial dental calculus.

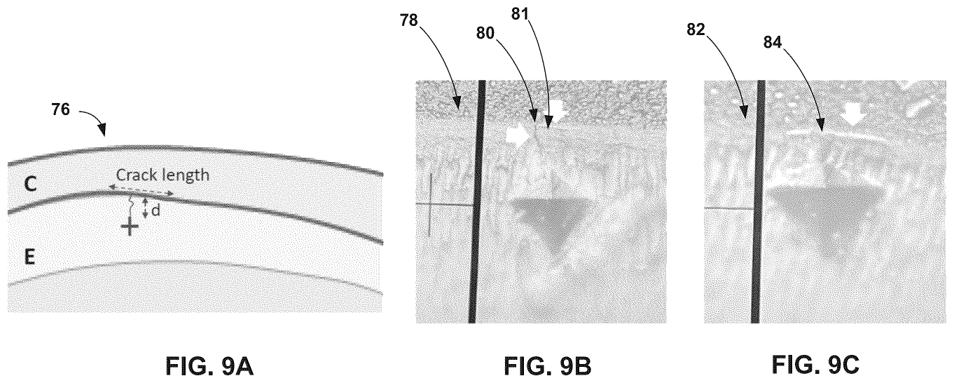

FIG. 9A is a diagram of a crack that can occur between the layers within calculus. Layers of calculus that are formed on a tooth or dental substrate may have relative weaknesses at the interface of the different layers. For example, calculus 76 may have different layers E and C. One or more cracks that run along the weak interfaces can be induced by cracks orthogonal to the layer direction created by an indenter. The interfacial crack length and/or depth can thus characterize the adhesive strength of the different layers in the calculus.

FIGS. 9B and 9C are images of example interfacial cracks in respective different artificial calculus samples. As shown in the calculus 78 of FIG. 9B, crack 80 has been generated orthogonal to the direction of the calculus layers. Crack 82 was also formed in the direction of the layer. As shown in the calculus 82 of FIG. 9C, crack 84 has been generated in the direction of the calculus layers. These types of interfacial cracks can be generated in natural calculus and in artificial calculus to measure their adhesive strength.

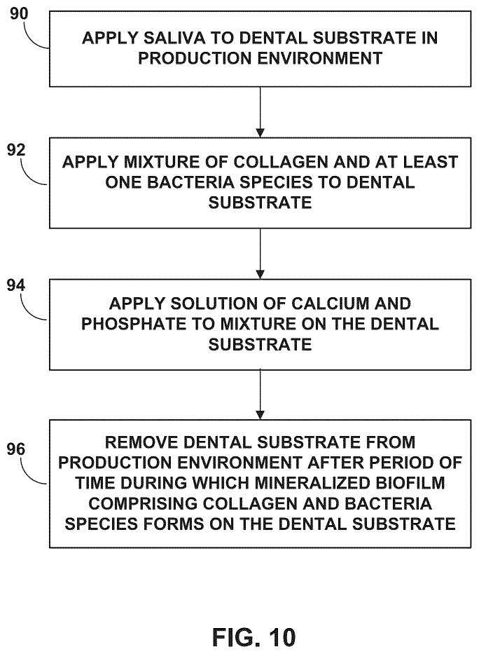

FIG. 10 is a flow diagram of an example technique for creating an artificial dental calculus model according to this disclosure. As shown in the example of FIG. 10, multiple steps may be used to create artificial dental calculus, such as in vivo calculus. The dental substrate may be selected as the foundation for the calculus to grow during these steps. These steps will be described as being performed by a user, but these steps may be performed by a machine or with robotic assistance in other examples.

In one example, a user applies saliva to the dental substrate in a production environment (90). This saliva may be applied to a dog tooth from which natural plaque and/or calculus has been removed or to an artificial substrate. The saliva may be left to soak on the dental substrate for a period of time that can range from minutes to several hours. One example soaking period may be approximately 4 hours.

Next, the user may apply a mixture of collagen and at least one bacterial species to the dental substrate (92). Although collagen can be used in some examples, other materials may be used as the extracellular support material instead of, or in addition to, collagen. Example bacterial species that may be applied include Neisseria canis and Frederiksenia canicola. In other example techniques, other single or combination of these or other bacterial species described herein may be applied. After the collagen and bacteria are applied, the user can apply a solution of calcium, phosphate, and, in some examples, a stabilizing agent to the mixture on the dental substrate (94). In some examples, the calcium and phosphate solution may include a stabilizing agent. The stabilizing agent may include agents such as polyacrylic acid, polyaspartic acid, polyallylamine hydrochloride, a polypeptide, as well as natural proteins such as statherin and fetuin and/or other chemicals or compounds. In some examples, the resulting mixture and solution may be left alone or undisturbed for a period of time, such as several hours to several days (e.g., 48 hours or 72 hours). In other examples, the solution of calcium, phosphate and a stabilizing agent may be reapplied during the rest period to continue to mineralize the artificial plaque on the dental substrate. In some examples, the steps of 92 and 94 may be referred to as one cycle. This cycle may be repeated one or more times, such as a total of 3 or 4 times in some examples. After the total number of cycles have been performed, the resulting artificial dental calculus on the dental substrate may be ready. Then, the user may remove the dental substrate from the production environment after the period of time during which mineralized biofilm comprising the collagen and bacterial species forms on the dental substrate (96).

In one specific example, the process of creating an in vitro dental calculus model includes applying saliva to a dental substrate for a duration from 1 minute to 6 hours in a production environment, wherein the dental substrate comprises a canine tooth removed of organically deposited plaque. In some examples, the saliva may be applied for a duration from about 30 minutes to about 90 minutes. The process then includes performing at least three cycles that each have a cycle period of at least 48 hours. Each cycle of the at least three cycles includes (1) applying a mixture of collagen and two bacterial species to the dental substrate, wherein the two bacterial species comprises Neisseria canis and Frederiksenia canicola, and (2) applying a solution comprising calcium, phosphate, and, in some examples, a stabilizing agent to the mixture on the dental substrate. Then, the process includes removing the dental substrate from the production environment after a period of time during which a mineralized biofilm comprising the collagen and the two bacterial species forms on the dental substrate.

Once the artificial dental calculus is created, it can be used to assess calculus treatments. In some examples, the user may apply one or more chemical treatments to the artificial dental calculus and assess whether the chemicals remove any calculus. In other examples, a mechanical removal process may be used that can include some removal medium. The removal medium may be a food substance that has been created to aid in the removal of calculus as the animal chews the food substance. In the testing setting, the dental substrate may be inserted into a chewing machine that generates mechanical forces on the artificial dental calculus with the food substrate. Using such processes, the artificial calculus can be analyzed to determine the efficacy of the calculus treatment. However, the use of the artificial calculus model may eliminate or reduce the need to rely on clinical studies and live animals to assess the efficacy of the treatment.

The process of creating an in vitro dental calculus can also be used to assess techniques and/or substances configured for the prevention of dental calculus. For example, for prevention, one or more chemical treatments can be applied to a substrate before or simultaneously (e.g., at about the same time in the process) as the substrate is exposed to the artificial calculus creation techniques. In the testing setting, for example, dental substrate with biofilm may be inserted into a chewing machine and be applied with calcium and phosphate solution, with or without stabilizing agents, and the chemical treatment simultaneously or in succession. The amount of calculus, or lack thereof, may indicate the effectiveness of the preventative treatment.

The following examples are described herein:

Example 1. A method for creating an in vitro dental calculus model, the method comprising: applying saliva to a dental substrate in a production environment; applying a mixture of an extracellular support material and at least one bacterial species to the dental substrate; applying a solution comprising calcium and phosphate to the mixture on the dental substrate; and removing the dental substrate from the production environment after a period of time during which a mineralized biofilm comprising the extracellular support material and the at least one bacterial species forms on the dental substrate.

Example 2. The method of example 1, wherein applying the saliva to the dental substrate comprises applying the saliva to the dental substrate for a duration from 1 minute to 6 hours.

Example 3. The method of any of examples 1 or 2, wherein a cycle comprises applying the mixture and applying the solution, and wherein the method comprises performing the cycle at least three times.

Example 4. The method of example 3, wherein each cycle comprises a cycle period of at least 48 hours.

Example 5. The method of any of examples 1 through 4, wherein the at least one bacterial species comprises at least two bacterial species.

Example 6. The method of example 5, wherein the at least two bacterial species comprise Neisseria canis and Frederiksenia canicola.

Example 7. The method of any of examples 1 through 6, wherein the at least one bacteria comprises no more than two bacterial species.

Example 8. The method of any of examples 1 through 7, wherein the at least one bacteria comprises an early colonizer selected to create an extracellular matrix of the mineralized biofilm.

Example 9. The method of any of examples 1 through 8, wherein the dental substrate comprises a canine tooth removed of organically deposited plaque.

Example 10. The method of any of examples 1 through 9, wherein the dental substrate comprises hydroxyapatite layer.

Example 11. The method of any of examples 1 through 10, wherein the saliva comprises at least one of natural canine saliva or an artificial saliva.

Example 12. The method of any of examples 1 through 11, further comprising: applying a dental treatment to the mineralized biofilm on the dental substrate, and analyzing an effectiveness of the dental treatment in removing at least a portion of the mineralized biofilm from the dental substrate.

Example 13. The method of any of examples 1 through 12, wherein the extracellular support material comprises collagen.

Example 14. The method of any of examples 1 through 13, wherein the solution comprises the calcium, phosphate, and a stabilizing agent.

Example 15. An in vitro dental calculus model, the dental calculus model comprising: a dental substrate; and a mineralized biofilm comprising an extracellular support material and at least one bacteria species.

Example 16. The dental calculus model of example 15, wherein the at least one bacteria species comprises at least two bacteria species.

Example 17. The dental calculus model of any of example 15 and 16, wherein the at least two bacteria species comprises Neisseria canis and Frederiksenia canicola.

Example 18. The dental calculus model of any of examples 15 through 17, wherein the at least one bacteria comprises no more than four bacteria species.

Example 19. The dental calculus model of any of examples 15 through 18, wherein the at least one bacteria comprises an early colonizer selected to create an extracellular matrix of the mineralized biofilm.

Example 20. The dental calculus model of any of examples 15 through 19, wherein the dental substrate comprises a canine tooth removed of organically deposited plaque.

Example 21. The dental calculus model of any of examples 15 through 20, wherein the dental substrate comprises hydroxyapatite layer.

Example 22. The dental calculus model of any of examples 15 through 21, wherein the mineralized biofilm comprises an average thickness greater than 150 micrometers.

Example 23. The dental calculus model of any of examples 15 through 22, wherein the extracellular support material comprises collagen.

Example 24. A method for creating an in vitro dental calculus model, the method comprising: applying saliva to a dental substrate for a duration from 2 hours to 6 hours in a production environment, wherein the dental substrate comprises a canine tooth removed of organically deposited plaque; performing at least three cycles that each have a cycle period of at least 48 hours, wherein each cycle of the at least three cycles comprises: applying a mixture of collagen and two bacteria species to the dental substrate, wherein the two bacteria species comprises Neisseria canis and Frederiksenia canicola; applying a solution comprising calcium and phosphate to the mixture on the dental substrate; and removing the dental substrate from the production environment after a period of time during which a mineralized biofilm comprising the collagen and the two bacteria species forms on the dental substrate.

Various examples have been described. These and other examples are within the scope of the following claims.

What is claimed is:

1. A method for creating an in vitro dental calculus model, the method comprising:
applying saliva to a dental substrate in a production environment;
applying a mixture of an extracellular support material and at least one bacterial species to the dental substrate;
applying a solution comprising calcium and phosphate to the mixture on the dental substrate; and
removing the dental substrate from the production environment after a period of time during which a mineralized biofilm comprising the extracellular support material and the at least one bacterial species forms on the dental substrate.

2. The method of claim 1, wherein applying the saliva to the dental substrate comprises applying the saliva to the dental substrate for a duration from 1 minute to 6 hours.

3. The method of claim 1, wherein a cycle comprises applying the mixture and applying the solution, and wherein the method comprises performing the cycle at least three times.

4. The method of claim 3, wherein each cycle comprises a cycle period of at least 48 hours.

5. The method of claim 1, wherein the at least one bacterial species comprises at least two bacterial species.

6. The method of claim 5, wherein the at least two bacterial species comprise Neisseria canis and Frederiksenia canicola.

7. The method of claim 1, wherein the at least one bacteria comprises no more than two bacterial species.

8. The method of claim 1, wherein the at least one bacteria comprises an early colonizer selected to create an extracellular matrix of the mineralized biofilm.

9. The method of claim 1, wherein the dental substrate comprises a canine tooth removed of organically deposited plaque.

10. The method of claim 1, wherein the dental substrate comprises hydroxyapatite layer.

11. The method of claim 1, wherein the saliva comprises at least one of natural canine saliva or an artificial saliva.

12. The method of claim 1, further comprising:
applying a dental treatment to the mineralized biofilm on the dental substrate, and
analyzing an effectiveness of the dental treatment in removing at least a portion of the mineralized biofilm from the dental substrate.

13. The method of claim 1, wherein the extracellular support material comprises collagen.

14. The method of claim 1, wherein the solution comprises the calcium, phosphate, and a stabilizing agent.

15. An in vitro dental calculus model, the dental calculus model comprising:
a dental substrate; and
a mineralized biofilm comprising an extracellular support material and at least one bacteria species.

16. The dental calculus model of claim 15, wherein the at least one bacteria species comprises at least two bacteria species.

17. The dental calculus model of claim 15, wherein the at least two bacteria species comprises Neisseria canis and Frederiksenia canicola.

18. The dental calculus model of claim 15, wherein the at least one bacteria comprises no more than four bacteria species.

19. The dental calculus model of claim 15, wherein the at least one bacteria comprises an early colonizer selected to create an extracellular matrix of the mineralized biofilm.

20. The dental calculus model of claim 15, wherein the dental substrate comprises a canine tooth removed of organically deposited plaque.

21. The dental calculus model of claim 15, wherein the dental substrate comprises hydroxyapatite layer.

22. The dental calculus model of claim 15, wherein the mineralized biofilm comprises an average thickness greater than 150 micrometers.

23. The dental calculus model of claim 15, wherein the extracellular support material comprises collagen.

24. A method for creating an in vitro dental calculus model, the method comprising:
applying saliva to a dental substrate for a duration from 2 hours to 6 hours in a production environment, wherein the dental substrate comprises a canine tooth removed of organically deposited plaque;
performing at least three cycles that each have a cycle period of at least 48 hours, wherein each cycle of the at least three cycles comprises:
applying a mixture of collagen and two bacteria species to the dental substrate, wherein the two bacteria species comprises Neisseria canis and Frederiksenia canicola;
applying a solution comprising calcium and phosphate to the mixture on the dental substrate; and
removing the dental substrate from the production environment after a period of time during which a mineralized biofilm comprising the collagen and the two bacteria species forms on the dental substrate.
